(19) United States
(12) Patent Application Publication
(10) Pub. No.: US 2016/0331295 A1
(43) Pub. Date: Nov. 17, 2016

(54) DETECTION OF A TRAUMATIC BRAIN INJURY WITH A MOBILE DEVICE

(71) Applicant: INTERNATIONAL BUSINESS MACHINES CORPORATION, Armonk, NY (US)

(72) Inventors: JAMES R. KOZLOSKI, NEW FAIRFIELD, CT (US); MARK C. H. LAMOREY, WILLISTON, VT (US); CLIFFORD A. PICKOVER, YORKTOWN HEIGHTS, NY (US); JOHN J. RICE, MOHEGAN LAKE, NY (US)

(21) Appl. No.: 14/745,492

(22) Filed: Jun. 22, 2015

Related U.S. Application Data
(63) Continuation of application No. 14/709,575, filed on May 12, 2015.

(51) Int. Cl. A61B 5/00; A61B 5/11; A42B 3/04

(52) U.S. Cl. CPC A61B 5/4064; A42B 3/0433; A61B 5/6803; A61B 5/7275; A61B 5/1121; A61B 5/6898; A61B 2562/0219; A61B 2503/10

(57) ABSTRACT

Embodiments include method, systems and computer program products for detecting a traumatic brain injury using a mobile device. Aspects include monitoring interactions of a user with a user interface of the mobile device during a time period and creating graphical representations of the interactions for one or more intervals during the time period. Aspects further include assigning a category to each of the graphical representations and creating an alert when a change in the assigned category of the graphical representations is detected.

### DOMESTIC PRIORITY

This application is a continuation of U.S. application Ser. No. 14/709,575; Attorney Docket: YOR920150161US1; Filed: May 12, 2015; which is related U.S. application Ser. No. 14/709,574; Attorney Docket: YOR920150162US1; Filed: May 12, 2015; U.S. application Ser. No. 14/709,572; Attorney Docket: YOR920150163US1; Filed: May 12, 2015; U.S. application Ser. No. 14/709,570; Attorney Docket: YOR920150164US1; Filed: May 12, 2015; U.S. application Ser. No. 14/709,563; Attorney Docket: YOR920150165US1; Filed: May 12, 2015; U.S. application Ser. No. 14/709,568; Attorney Docket: YOR920150167US1; Filed: May 12, 2015; U.S. application Ser. No. 14/709,564; Attorney Docket YOR920150168US1; Filed: May 12, 2015; U.S. application Ser. No. 14/664,987; Filed Mar. 23, 2015; Attorney Docket No.: YOR920150038US1; U.S. application Ser. No. 14/664,989; Filed: Mar. 23, 2015; Attorney Docket No.: YOR920150039US1; and U.S. application Ser. No. 14/664,991; Filed: Mar. 23, 2015; Attorney Docket No.: YOR920150040US1, the contents of each of which are herein incorporated by reference in their entirety.

### BACKGROUND

The present disclosure relates to the detection of a traumatic brain injury using a mobile device, and more specifically, to methods, systems and computer program products for the detection of a traumatic brain injury through the observation of a user's interaction with a user interface of a mobile device.

A concussion is a type of traumatic brain injury that is caused by a blow to the head that shakes the brain inside the skull due to linear or rotational accelerations. Recently, research has linked concussions to a range of health problems, from depression to Alzheimer's, along with a range of brain injuries. Unlike severe traumatic brain injuries, which result in lesions or bleeding inside the brain and are detectable using standard medical imaging, a concussion is often invisible in brain tissue, and therefore only detectable by means of a cognitive change, where that change is measurable by changes to brain tissue actions, either neurophysiological or through muscle actions caused by the brain and the muscles resulting effects on the environment, for example, speech sounds.

### SUMMARY

In accordance with an embodiment, a method for detecting a traumatic brain injury using a mobile device is provided. The method includes monitoring interactions of a user with a user interface of the mobile device during a time period and creating graphical representations of the interactions for one or more intervals during the time period. The method further includes assigning a category to each of the graphical representations and creating an alert when a change in the assigned category of the graphical representations is detected.

In accordance with another embodiment, a mobile device for detecting a traumatic brain injury includes a processor and a user interface, the processor being configured to perform a method. The method includes monitoring interactions of a user with a user interface of the mobile device during a time period and creating graphical representations of the interactions for one or more intervals during the time period. The method further includes assigning a category to each of the graphical representations and creating an alert when a change in the assigned category of the graphical representations is detected.

In accordance with a further embodiment, a computer program product for detecting a traumatic brain injury using a mobile device includes a non-transitory storage medium readable by a processing circuit and storing instructions for execution by the processing circuit for performing a method. The method includes monitoring interactions of a user with a user interface of the mobile device during a time period and creating graphical representations of the interactions for one or more intervals during the time period. The method further includes assigning a category to each of the graphical representations and creating an alert when a change in the assigned category of the graphical representations is detected.

### BRIEF DESCRIPTION OF THE DRAWINGS

The subject matter which is regarded as the invention is particularly pointed out and distinctly claimed in the claims at the conclusion of the specification. The forgoing and other features, and advantages of the invention are apparent from the following detailed description taken in conjunction with the accompanying drawings in which:

FIG. 1 is a block diagram illustrating one example of a processing system for practice of the teachings herein;

FIG. 2 is a block diagram illustrating a helmet in accordance with an exemplary embodiment;

FIG. 3 is a block diagram illustrating a system for detecting a traumatic brain injury using a mobile device in accordance with an exemplary embodiment;

FIG. 4 is a flow diagram of a method for detecting a traumatic brain injury using a mobile device in accordance with an exemplary embodiment;

FIG. 5 is a flow diagram of another method for detecting a traumatic brain injury through using a mobile device in accordance with an exemplary embodiment and

FIG. 6 is a flow diagram of method for detecting a traumatic brain injury through using a mobile device and a helmet in accordance with an exemplary embodiment.

### DETAILED DESCRIPTION

In accordance with exemplary embodiments of the disclosure, methods, systems and computer program products for detecting a traumatic brain injury using a mobile device are provided. In exemplary embodiments, an individual, which may be wearing a helmet, is using a mobile device that has a user interface. The mobile device is configured to monitor the interaction of the user with the user interface and to construct a graphical representation of the interactions. In exemplary embodiments, the graphical representation can be a directed graph in which state of the user interface is represented by a node and in which each transition between states is represented by an edge in the directed graph. The directed graph is analyzed over time and changes in the characteristics of the directed can be correlated with transitions in the cognitive state of the user. In one example, a baseline directed graph is constructed based on the observation of the user's interaction with the user interface while the user is in a normal cognitive state. In exemplary embodiments, the usage of the user interface, and the directed graph derived therefrom, is compared to the baseline directed graph and deviations from the baseline directed graph can be used to indicate changes in the user's cognitive state, i.e., that the user has suffered a traumatic brain injury.

In exemplary embodiments, the mobile device may be configured to communicate with a helmet that includes one or more sensors. In exemplary embodiments, the sensors may include one or more accelerometers, gyroscopes, or the like. In one embodiment, the outputs of the sensors are provided to a processor that monitors one or more physical movements or actions of the user. The processor is configured to monitor the output of the sensors to determine if a serious impact has occurred. As used herein, a serious impact is an impact that may cause a traumatic brain injury. In exemplary embodiments, the mobile device and the helmet may be configured to communicate with each other, or with a separate processing system, to correlate indications of a traumatic brain injury from the helmet and the mobile device. This correlation may be used to increase a confidence level associated with the alert that a user suffered a traumatic brain injury.

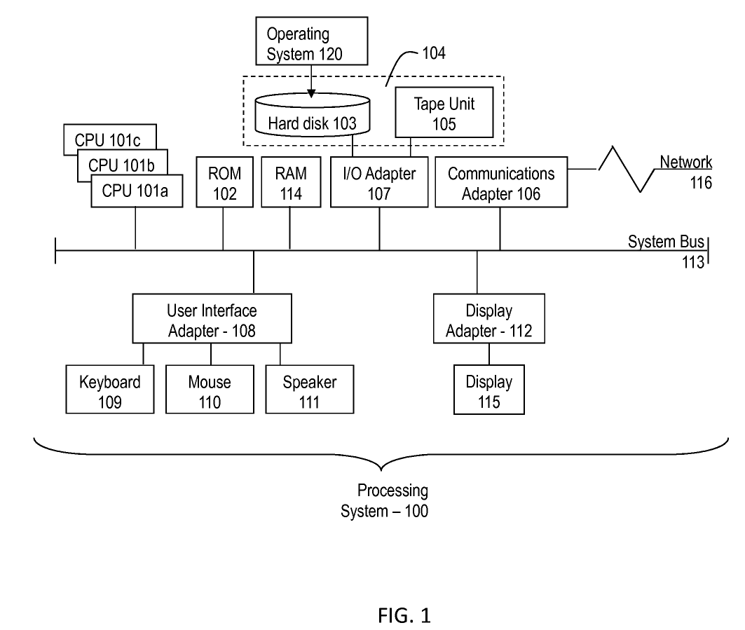

Referring to FIG. 1, there is shown an embodiment of a processing system 100 for implementing the teachings herein. In this embodiment, the system 100 has one or more central processing units (processors) 101a, 101b, 101c, etc. (collectively or generically referred to as processor(s) 101). In one embodiment, each processor 101 may include a reduced instruction set computer (RISC) microprocessor. Processors 101 are coupled to system memory 114 and various other components via a system bus 113. Read only memory (ROM) 102 is coupled to the system bus 113 and may include a basic input/output system (BIOS), which controls certain basic functions of system 100.

FIG. 1 further depicts an input/output (I/O) adapter 107 and a network adapter 106 coupled to the system bus 113. I/O adapter 107 may be a small computer system interface (SCSI) adapter that communicates with a hard disk 103 and/or tape storage drive 105 or any other similar component. I/O adapter 107, hard disk 103, and tape storage device 105 are collectively referred to herein as mass storage 104. Operating system 120 for execution on the processing system 100 may be stored in mass storage 104. A network adapter 106 interconnects bus 113 with an outside network 116 enabling data processing system 100 to communicate with other such systems. A screen (e.g., a display monitor) 115 is connected to system bus 113 by display adaptor 112, which may include a graphics adapter to improve the performance of graphics intensive applications and a video controller. In one embodiment, adapters 107, 106, and 112 may be connected to one or more I/O busses that are connected to system bus 113 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 113 via user interface adapter 108 and display adapter 112. A keyboard 109, mouse 110, and speaker 111 all interconnected to bus 113 via user interface adapter 108, which may include, for example, a Super I/O chip integrating multiple device adapters into a single integrated circuit.

Thus, as configured in FIG. 1, the system 100 includes processing capability in the form of processors 101, storage capability including system memory 114 and mass storage 104, input means such as keyboard 109 and mouse 110, and output capability including speaker 111 and display 115. In one embodiment, a portion of system memory 114 and mass storage 104 collectively store an operating system such as the AIX® operating system from IBM Corporation to coordinate the functions of the various components shown in FIG. 1.

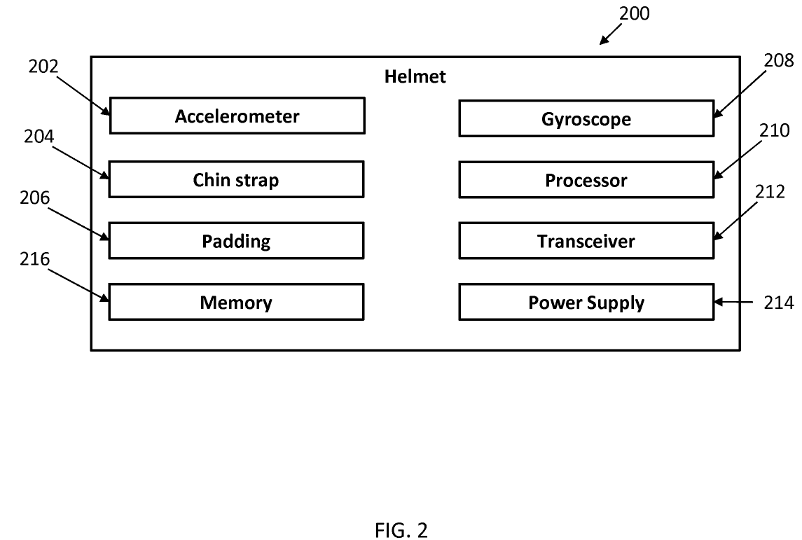

Referring now to FIG. 2, a block diagram illustrating a helmet 200 in accordance with an exemplary embodiment is shown. The term “helmet” may include, but is not intended to be limited to, a football helmet, a helmet worn by a soldier, a motorcycle helmet or the like. In exemplary embodiments, the helmet 200 includes one or more of the following: an accelerometer 202, a chin strap 204, a padding 206, a gyroscope 208, a processor 210, a transceiver 212, a power supply 214 and a memory 216. In exemplary embodiments, the padding 206 of the helmet 200 may include either or both of internal padding or external padding that can have one or more adjustable parameters. In exemplary embodiments, the power supply 214 may be a battery configured to provide power to one or more of the accelerometer 202, the gyroscope 208, the processor 210 and the transceiver 212. In one embodiment, the processor 210 is configured to receive an output from one or more of the accelerometer 202 and the gyroscope 208 and to determine if the user of the helmet has suffered a severe impact. For example, the processor 210 may determine that the user of the helmet has suffered a severe impact if the acceleration and/or rotation experienced by the helmet exceed one or more threshold values.

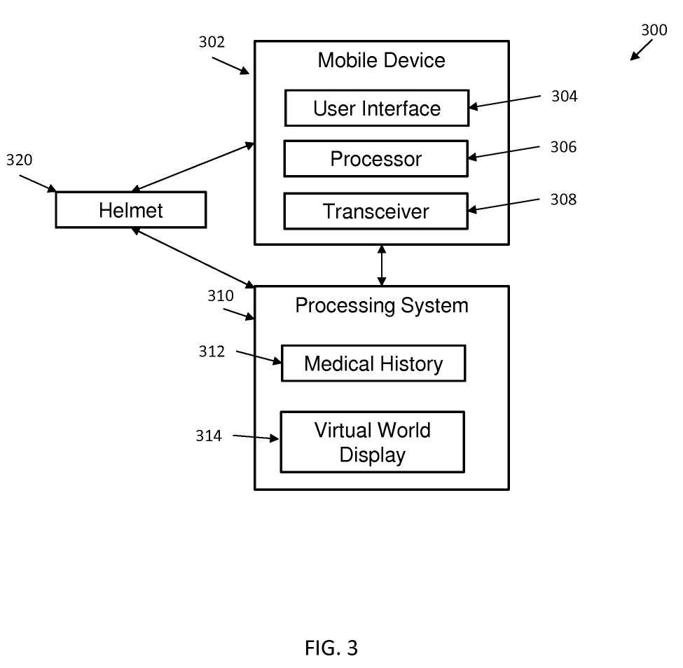

Referring now to FIG. 3, a block diagram illustrating a system 300 for detecting a traumatic brain injury using a mobile device in accordance with an exemplary embodiment is shown. In exemplary embodiments, the system 300 includes a mobile device 302 that includes a user interface 304, a processor 306 and a transceiver 308. The mobile device 300 may be any suitable mobile device including, but not limited to, a smartphone, a tablet, a laptop, or the like. The processor 306 of the mobile device 302 receives inputs from the user interface 304 and responsively controls the operation of the mobile device 302. In addition, the processor 306 may store data regarding the operation of the mobile device 302 and the user interface 304 in a memory and perform analysis on this data. The transceiver 308 of the mobile device 302 is configured to communicate with one or more of a helmet 320 and a processing system 310.

In exemplary embodiment, the mobile device 302 is configured to monitor the interactions of a user with the user interface 304 and to construct a graphical representation of these interactions. In exemplary embodiments, the graphical representation is a directed graph in which state of the user interface is represented by a node, and in which each transition between states is represented by an edge in the directed graph. The analysis of the directed graph can include analyzing one or more characteristics of the directed graph, which may include a diameter of the directed graph, a number of loops in the directed graph, and a topological motif of directed graph or subgraphs selected based on other contextual measures.

In exemplary embodiments, the analysis of the directed graph may include creating one or more category labels based on the type of usage. For example, the directed graph may be analyzed under when the user is operating in a known cognitive state, i.e., under normal usage conditions and a baseline directed graph is constructed. In exemplary embodiments the analyzing the directed graph may include categorizing the directed graph into one or more categories based on the one or more characteristics of the directed graph. The directed graph may be constantly or periodically updated based on the interactions of the user with the user interface and changes in the determined category of the directed graph can be used to create alerts. In one embodiment, a change in the category of the directed graph, or in one of the characteristics of the directed graph exceeding a threshold, may be indicative of a change in the cognitive state of the user and may suggest that the user may have suffered a traumatic brain injury. In exemplary embodiments, the mobile device 302 is configured to transmit alerts that the user may have suffered a traumatic brain injury via the transceiver 308.

As illustrated, the system 300 includes a helmet 320, such as the one shown and described above with reference to FIG. 2, and a processing system 310, such as the one shown and described above with reference to FIG. 1. In exemplary embodiments, when a change in category of the directed graph is detected, a secondary correlation check with helmet acceleration/rotation data can be performed using data from the helmet 320. In exemplary embodiments, this secondary correlation may be performed by the mobile device 302, the helmet 320 or by the processing system 310. If a change in the graph category is correlated with recent acceleration/rotation of the helmet 320, and the graph category is indicative a decrease in cognitive function, a traumatic brain injury alert can be created. In exemplary embodiments, the alert may include an identification of the user and a confidence level associated with the risk of traumatic brain injury, which may include the data obtained by the helmet 320 and/or the mobile device 302.

In exemplary embodiments, the processing system 310 is configured to communicate with the helmet 320 and may also be configured to store the medical history 312 of the users of the helmets 320. In exemplary embodiments, the medical history 312 of the users of the helmets 320 may be used by the helmet 320 or the mobile device 302 in determining when to create an alert of a possible traumatic brain injury. In addition, the processing system 310 may include a virtual world display 314 that is configured to provide a display a real-time status of each of the users based on data received from the helmet 320 and/or the mobile devices 302. In exemplary embodiments, the status may include, the category of play of each user, any indications that the user may have suffered a traumatic brain injury, a duration of play of the user, a duration that the user has been in the current category of play, or the like.

In exemplary embodiments, the user's history of collision or medical concerns may be used to determine a traumatic brain injury risk assessment, either by the embedded processors in the helmet 320 or mobile device 302 or by the processing system 310. In addition, the helmet 320 and/or mobile device 302 may be configured to provide a real-time feed of the user's cognitive state to increase the confidence level of the need for a particular alert or indication. In exemplary embodiments, an aggregate indication may be used by the processing system 310 to summarize an overall state of a group of players. This may also help to potentially identify areas of risk in the dynamics of player-player interaction, overly aggressive players, playing field conditions, etc. In exemplary embodiments, an automatic feed from a user's history of collision or medical concerns may also be provided to a processor of helmet 320 or mobile device 302 in order to update an impact risk model used by the helmet 320 or mobile device 302. In addition, the processing system 310 may receive a real-time feed of the user's cognitive state, which can be used to update the risk models used by the processing system 310. The risk models may also be sent to the virtual world display 314 of the game and players, which allows the sports staff health professionals to visualize the nature of potential problems.

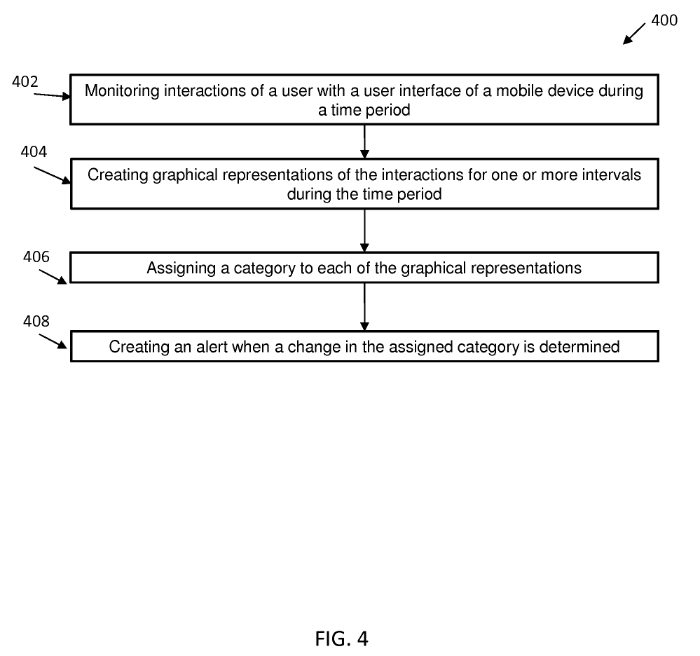

Referring now to FIG. 4, a flow diagram of a method 400 for detecting a traumatic brain injury using a mobile device in accordance with an exemplary embodiment is shown. As shown at block 402, the method 400 includes monitoring interactions of a user with a user interface of a mobile device during a time period. In exemplary embodiments, the user interface may include a touch screen device, a multi-touch device, or the like. Next, as shown at block 404, the method 400 includes creating graphical representations of the interactions for one or more intervals during the time period. In one example, the time period may be thirty minutes and the time intervals may be five or ten minutes each. In exemplary embodiments, the graphical representations can be directed graphs in which a state of the user interface is represented by a node and in which transitions between states of the user interface are represented edges in the directed graph. The method 400 also includes assigning a category to each of the graphical representations, as shown at block 406. In exemplary embodiments, the graphical representations are assigning to a category based on an analysis of the directed graph, which can include analyzing one or more characteristics of the directed graph, which may include a diameter of the directed graph, a number of loops in the directed graph, and a topological motif of directed graph or subgraphs selected based on other contextual measures. Next, as shown at block 408, the method 400 includes creating an alert when a change in the assigned category of the graphical representations is determined. In exemplary embodiments, a change in the assigned category of the graphical representations can be correlated with a change in the cognitive state of the user.

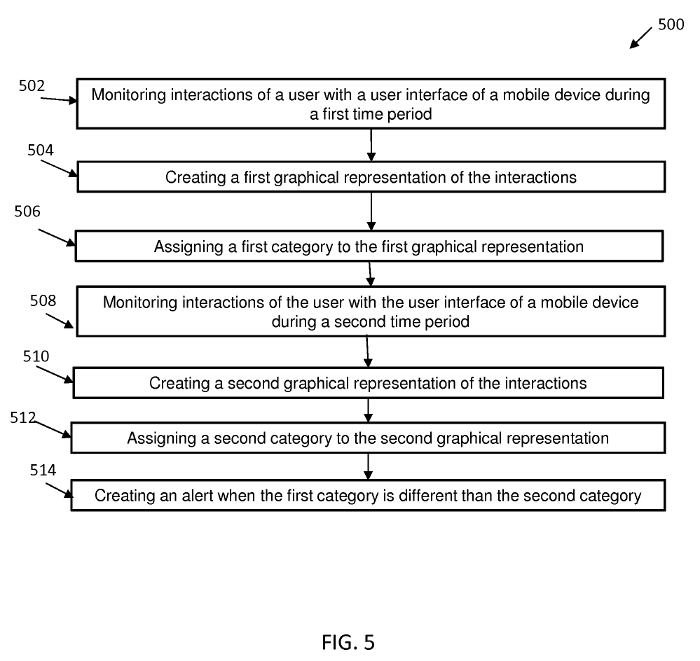

Referring now to FIG. 5, a flow diagram of another method 500 for detecting a traumatic brain injury using a mobile device in accordance with an exemplary embodiment is shown. As shown at block 502, the method 500 includes monitoring interactions of a user with a user interface of a mobile device during a first time period. Next, as shown at block 504, the method 500 includes creating a first graphical representation of the interactions during the first time period. The method 500 also includes assigning a first category to the first graphical representation, as shown at block 506. In exemplary embodiments, the graphical representations are assigning to a category based on an analysis of one or more characteristics of the first directed graph.

Continuing with reference to FIG. 5, the method 500 also includes monitoring interactions of the user with the user interface of the mobile device during a second time period, as shown at block 508. Next, as shown at block 510, the method 500 includes creating a second graphical representation of the interactions during the second time period. In exemplary embodiments, the first and second graphical representations can be directed graphs in which a state of the user interface is represented by a node and in which transitions between states of the user interface are represented edges in the directed graphs. The method 500 includes assigning a second category to the second graphical representation, as shown at block 512. In exemplary embodiments, the graphical representations are assigning to a category based on an analysis of one or more characteristics of the second directed graph. Next, as shown at block 514, the method includes creating an alert when the first category is different than the second category. In exemplary embodiments, the alert may include an identification of the user and an identification of the first category and the second category. In one embodiment, the first and second time periods are consecutive time intervals. In another embodiment, the first time period is a period when the user is operating in a known cognitive state and the second time period is when the user is operating in an unknown cognitive state.

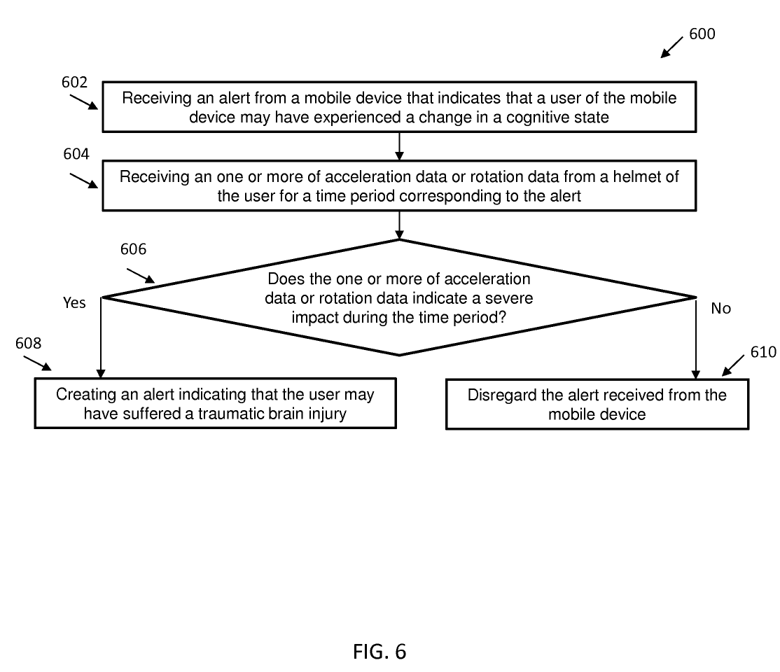

Referring now to FIG. 6, a flow diagram of another method 600 for detecting a traumatic brain injury using a mobile device and a helmet in accordance with an exemplary embodiment is shown. As shown at block 602, the method 600 includes receiving an alert from a mobile device that indicates that a user of the mobile device may have experienced a change in a cognitive state. Next, as shown at block 604, the method 600 includes receiving one or more of acceleration data or rotation data from a helmet of the user for a time period corresponding to the alert. As shown at decision block 606, the method 600 includes determining if the one or more of acceleration data or rotation data indicate a severe impact during the time period. In exemplary embodiments, the determination if the acceleration data or rotation data indicates a severe impact during the time period includes determining that either or both of the acceleration or rotation experienced by the helmet exceeds a threshold level. If the one or more of acceleration data or rotation data indicate a severe impact during the time period, then the method 600 proceeds to block 608 and includes create an alert indicating that the user may have suffered a traumatic brain injury. Otherwise, the method 600 proceeds to block 610 and disregards the alert received from the mobile device.

In exemplary embodiments, the mobile device may be configured to create graphical representations of the interactions for one or more intervals during the time period and to assign a category to each of the graphical representations. In other embodiments, the mobile device may monitor the interactions of the user with a user interface and provide those interactions to a separate processing system. In these embodiments, the separate processing system may be configured to create graphical representations of the interactions for one or more intervals during the time period and to assign a category to each of the graphical representations.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method for detecting a traumatic brain injury using a mobile device, the method comprising:
monitoring interactions of a user with a user interface of the mobile device during a time period;
creating graphical representations of the interactions for one or more intervals during the time period;
assigning a category to each of the graphical representations; and
creating an alert when a change in the assigned category of the graphical representations is detected.

2. The method of claim 1, wherein the graphical representations comprise directed graphs in which a state of the user interface is represented by a node and in which transitions between states of the user interface are represented by edges.

3. The method of claim 2, wherein the graphical representations are assigned to the category based on an analysis of the directed graphs, which includes analyzing one or more characteristics of the directed graphs.

4. The method of claim 3, wherein the one or more characteristics of the directed graphs include at least one of a diameter of the directed graphs, a number of loops in the directed graphs, and a topological motif of the directed graphs.

5. The method of claim 1, wherein the change in the assigned category of the graphical representations is indicative of a change in a cognitive state of the user.

6. The method of claim 1, further comprising:
receiving one or more of an acceleration data or a rotation data from a helmet of the user for a portion of the time period corresponding to the alert;
determining if the one or more of the acceleration data or the rotation data indicates that the helmet experienced a severe impact during the time period; and
creating an indication that the user may have suffered a traumatic brain injury based on determining that the helmet experienced a severe impact during the time period.

7. The method of claim 6, further comprising correlating the change in the assigned category of the graphical representations with the indication that the user may have suffered the traumatic brain injury and including a quantification of a risk of traumatic brain injury in the alert.
